(19) United States
(12) Patent Application Publication
(10) Pub. No.: US 2012/0156096 A1
(43) Pub. Date: Jun. 21, 2012

(54) Medical Sterilization Container Shape Memory Alloy (SMA)Valve

(76) Inventors: Kraig Herman Allen, Leesburg, IN (US); Jon D. Shoemaker, Leesburg, IN (US); Gary T. Dane, Bow, NH (US); Reese Myers, Columbia City, IN (US)

(21) Appl. No.: 13/330,061

(22) Filed: Dec. 19, 2011

Related U.S. Application Data
(60) Provisional application No. 61/424,310, filed on Dec. 17, 2010.

(51) Int. Cl. A61L 2/04

(52) U.S. Cl. 422/38; 422/300

(57) ABSTRACT

A medical sterilization container valve and method of making the same is provided. The medical sterilization container valve includes a valve housing affixed to a medical sterilization container. A first valve portion is attached to the valve housing. A second valve portion is proximate to the first valve portion, wherein the second valve portion is movable between at least a first position and a second position. A shape metal alloy (SMA) device is connected with the second valve portion, wherein the SMA device moves the second valve portion between the first and second positions.

### CROSS REFERENCE TO RELATED APPLICATION

This application claims benefit of U.S. Provisional Application Ser. No. 61/424,310, entitled, “Medical Sterilization Container Shape Memory Alloy (SMA) Valve” filed Dec. 17, 2010, the entire disclosure of which is incorporated herein by reference.

### FIELD OF THE DISCLOSURE

The present disclosure is generally related to medical sterilization container valves and more particularly is related to a medical sterilization container shape memory alloy valve.

### BACKGROUND OF THE DISCLOSURE

Articles such as medical instruments and the like are usually sterilized in an autoclave in which the articles are exposed to high-pressure saturated steam for a relatively brief interval. Unless the articles are to be used immediately and in close proximity to the autoclave, it is desirable to sterilize the articles while they are inside a valved container as described, for example, in U.S. Pat. No. 4,748,003 and in U.S. Pat. No. 5,097,865.

During the sterilization process, the valves open under the influence of high-pressure steam in the autoclave exposing the contents of the container to the hot steam. At the end of the sterilization cycle, when the pressure in the autoclave outside the container is returned to normal, i.e. atmospheric pressure, the valves close so that when the container is removed from the autoclave, the now sterilized articles are maintained in a completely sealed sterile environment until they are needed.

The sterilization container described above has pressure-actuated valves in the top and bottom walls of the sterilization container. Each valve has a large valve opening and a closure therefor, the latter being supported by a bellows capsule mounted inside the container. A return spring mechanism normally maintains the valve closure in its closed position. However, when the pressure outside the container exceeds that within the container by a few pounds per square inch, the force on the valve closure exceeds that exerted by the return spring with the result that the valve closure opens sufficiently to allow high-pressure steam to enter the container. That steam collapses the bellows, which thereupon moves the valve closure to its fully open position. Both valves being open, high-pressure steam can sweep through the container and sterilize the articles therein.

When the pressure inside the autoclave returns to normal after completion of the sterilization cycle, the return spring moves the valve closure of each valve to its closed position thereby sealing the container. As also described, pressure equalization occurs through a special filter member mounted in the container wall so that a sterile environment at ambient pressure is maintained in the container until the container is opened to remove the articles therefrom.

The conventional valves do have certain drawbacks. First and foremost, the bellows capsule has a relatively small diameter in relation to the valve opening and a large length-to-diameter ratio, e.g. 1.3 in./1 in.=1.3. This means that when the valve member starts to open in response to a given pressure differential outside and inside the container and the bellows is collapsed lengthwise, there is a relatively large reduction in the volume of the bellows, i.e. in excess of 20%, and a correspondingly large increase in gas pressure inside the bellows which resists further opening of the valve member.

To avoid this problem, the bellows capsule in the prior value has to be evacuated. This necessitates the use of an elaborate return spring mechanism in order to overcome the increased back pressure and close the valve. Also, the requirement for a bellows that must be evacuated makes the valve somewhat more difficult and expensive to manufacture. In addition, sometimes a small leak may develop in the bellows so that air enters the bellows. Resultantly, during the next sterilization cycle, when the valve member begins to open, that air will be compressed, effectively increasing the spring constant of the bellows so that the valve member does not open as much for a given pressure differential outside and inside the container.

Thus, a heretofore unaddressed need exists in the industry to address the aforementioned deficiencies and inadequacies.

### SUMMARY OF THE DISCLOSURE

Embodiments of the present disclosure provide a system for a medical sterilization container valve. Briefly described, in architecture, one embodiment of the system, among others, can be implemented as follows. The medical sterilization container valve has a valve housing affixed to a medical sterilization container. A first valve portion is attached to the valve housing. A second valve portion is proximate to the first valve portion, wherein the second valve portion is movable between at least a first position and a second position. A shape metal alloy (SMA) device is connected with the second valve portion, wherein the SMA device moves the second valve portion between the first and second positions.

The present disclosure can also be viewed as providing a method of utilizing a valve within a medical sterilization container. In this regard, one embodiment of such a method, among others, can be broadly summarized by the following steps: affixing a valve housing to a medical sterilization container, wherein the valve housing has a first valve portion attached to the valve housing, and a second valve portion proximate to the first valve portion, wherein the second valve portion is movable between at least a first position where the first and second valve portions are in contact, and a second position where the first and second valve portions are not in contact; connecting a shape metal alloy (SMA) device into the second valve portion; and subjecting the SMA device to a thermal energy, thereby moving the second valve portion between the first and second positions with the SMA device.

The present disclosure can also be viewed as providing a system for sterilizing medical instruments and storing sterilized medical instruments. Briefly described, in architecture, one embodiment of the system, among others, can be implemented as follows. A medical sterilization container has a plurality of side walls and a cover, wherein the cover is openable to expose an interior portion of the medical sterilization container, wherein the interior portion is sized to house a quantity of medical instruments. A valve is positioned within the cover, wherein the valve has a valve housing, a first valve portion attached to the valve housing, and a second valve portion proximate to the first valve portion, wherein the second valve portion is movable between at least a first position and a second position. A shape metal alloy (SMA) device is connected with the second valve portion, wherein the SMA device moves the second valve portion between the first and second positions. An access path is positioned through the valve between an exterior of the medical sterilization container and the interior portion, wherein the access path is closed when the second valve portion is in the first position and open when the second valve portion is in the second position.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a cross-sectional illustration of a medical sterilization container having a medical sterilization container valve, in accordance with a first exemplary embodiment of the present disclosure.

FIG. 2 is a cross-sectional illustration of a medical sterilization container valve, in accordance with the first exemplary embodiment of the present disclosure.

FIG. 3 is a cross-sectional illustration of a medical sterilization container valve, in accordance with the first exemplary embodiment of the present disclosure.

FIG. 4 is a cross-sectional illustration of a medical sterilization container valve within a medical sterilization container, in accordance with a second exemplary embodiment of the present disclosure.

FIG. 5 is a cross-sectional illustration of a medical sterilization container valve, in accordance with the second exemplary embodiment of the present disclosure.

FIG. 6 is a cross-sectional illustration of a medical sterilization container valve, in accordance with a third exemplary embodiment of the present disclosure.

FIG. 7 is a cross-sectional illustration of a medical sterilization container valve, in accordance with a fourth exemplary embodiment of the present disclosure.

FIG. 8 is a cross-sectional illustration of a medical sterilization container valve, in accordance with the fourth exemplary embodiment of the present disclosure.

FIG. 9 is a flowchart illustrating a method of utilizing a valve within a medical sterilization container, in accordance with the first exemplary embodiment of the present disclosure.

### DETAILED DESCRIPTION

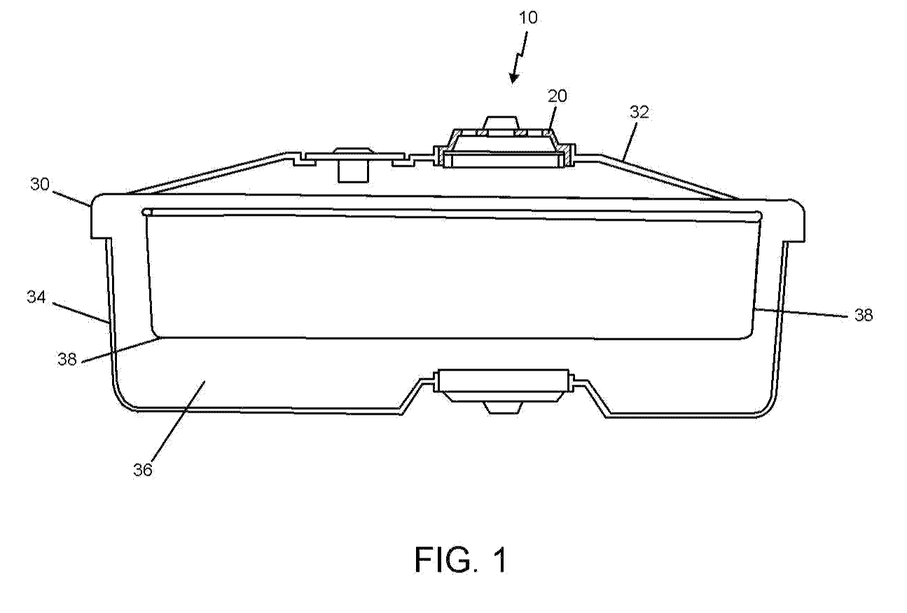

FIG. 1 is a cross-sectional illustration of a medical sterilization container 30 having a medical sterilization container valve 10, in accordance with a first exemplary embodiment of the present disclosure. The medical sterilization container valve 10 (hereinafter, “valve 10”), may be used with a medical sterilization container 30 that houses a plurality of medical instruments during a sterilization process, as well as before and after a sterilization process. The sterilization process may include subjecting the medical sterilization container to environments with high heat and high pressures, such as is commonly seen within an autoclave. Sterilization fluid may be passed over the medical sterilization container 30 and steam and/or another fluid may pass through the medical sterilization container 30. The valve 10 allows for the escape and entry of sterilization fluid to and from the medical sterilization container 30 as the temperature and pressure of the sterilization environment changes.

The valve 10 may be used with a medical sterilization container 30 having any size or shape. The medical sterilization container 30 is used to house medical instruments and other medical devices in between medical procedures. For example, the medical sterilization container 30 may be used to house operating or surgical devices during a sterilization process and until their use in an operation or surgical procedure. The sterilization processes that the medical sterilization container 30 is subjected to may include any type of chemical, pressure, heat, and/or energy-based sterilization process. Commonly, the sterilization process includes the use of an autoclave which subjects the medical sterilization container 30 to high temperatures, high pressures, and moist environments which may include a sterilization chemical. However, other sterilization processes may include the use of other devices or other sterilization techniques, such high-temperature environments and ultra-violet light screenings.

The valve 10 may be located within any part of the medical sterilization container 30. For example, as is shown in FIG. 1, the valve 10 may be integral with a cover 32 of the medical sterilization container 30, or within a base 34 of the medical sterilization container 30. The cover 32 may connect to the base 34 to create an interior portion 36 which houses a tray 38. The tray 38 may be sized to hold any quantity or type of medical instrument (not shown). It is noted that the cover 32 may connect to the base 34 with any type of design that will enclose the interior portion 36 and allow access to the interior portion 36. For example, the cover 32 may be affixed to the base 34 with a removable fastener or biased structure, wherein a user of the medical sterilization container 30 is able to engage and disengage the fastener or biased structure from the cover 32. Other variations to the medical sterilization container 30 are well-known within the art and are considered within the scope of the present disclosure.

The valve 10 includes a valve housing 20, which is positioned within a sidewall, the base 34, or a cover 32 of the medical sterilization container 30. For example, the valve housing 20 may be integrally mounted within a cover 32 of the medical sterilization container 30, as is shown. In this position, the valve 10 is positioned at an interface between an exterior of the medical sterilization container 30, such as any atmosphere external to the interior part of the medical sterilization container 30, and the interior portion 36 of the medical sterilization container 30. The valve 10 may be similarly located within any portion of the base 34, as may vary by design. The cover 32 may be removable from the base 34, thereby allowing access to the interior portion 36 to insert medical instruments or remove them therefrom. When the cover 32 is affixed to the base 34, the interior portion 36 may be substantially sealed from the external environment, whereby access to the interior portion 36 of the medical sterilization container 30 is through the valve 10, or another similar structure, such as a secondary valve.

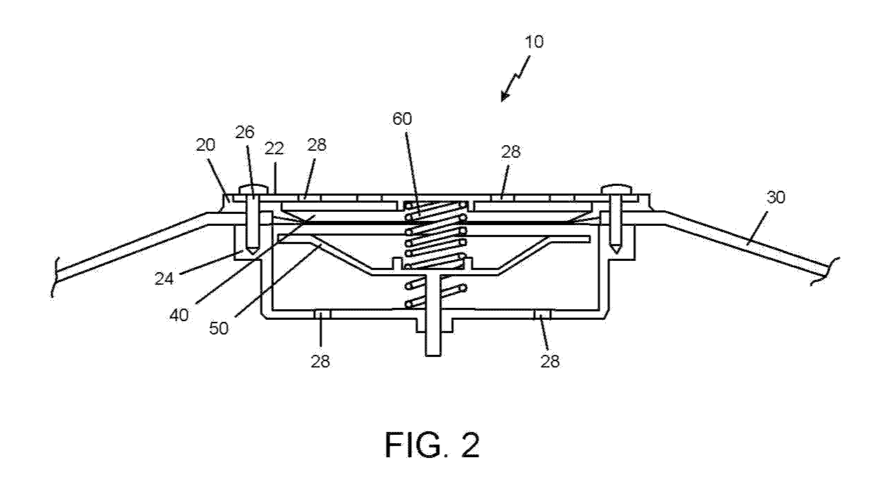

FIG. 2 is a cross-sectional illustration of a medical sterilization container valve 10, in accordance with the first exemplary embodiment of the present disclosure. The valve 10 includes a valve housing 20 affixed to a medical sterilization container 30. A first valve portion 40 may be attached to the valve housing 20 and a second valve portion 50 may be proximate to the first valve portion 40. The second valve portion 50 may be movable between at least a first position and a second position. A shape metal alloy (SMA) device 60 may be connected with the second valve portion 50. The SMA device 60 may move the second valve portion 50 between the first and second positions.

The valve 10 may be assembled and connected to the medical sterilization container 30 in a number of ways. The valve 10 includes a valve housing 20, which is positioned within a portion of the medical sterilization container 30. The valve housing 20 includes a first valve housing structure 22 and a second valve housing structure 24 which may be affixed together around the medical sterilization container 30. In other words, a portion of the medical sterilization container 30 may be located between the first and second valve housing structures 22, 24. For example, as is shown in FIG. 2, the first valve housing structure 22 may be positioned on one side of the medical sterilization container 30, whereas the second valve housing structure 24 is positioned on an opposing side of the medical sterilization container 30. A fastener 26 may be used to retain the first and second valve housing structures 22, 24 in place about the medical sterilization container 30. The fastener 26 may include any type of fastener, such as a threaded connector, a nut and bolt system, a rivet connection, or an adhesive connector. Additionally, the first and second valve housing structures 22, 24 may include threaded or snap connectors that engage with each other directly to secure the first and second valve housing structures 22, 24 together without the use of a separate fastener.

The valve housing 20 may be constructed from any type of material, and may be configured or designed in a number of different ways, and may include a plurality of additional features. For example, either or both of the first and second valve housing structures 22, 24 may include a plurality of perforations 28, which allow for gas, moisture, or other materials to penetrate therethrough. These perforations 28, which may include any type of hole, aperture, or other opening, may also allow a sterilization material, such as steam, gas, a chemical, or a heated substance to pass from the exterior of medical sterilization container 30 to the interior portion 36 (FIG. 1), when the valve 10 is open. Of course, the valve housing 20 may be sized accordingly to fit within new or existing medical sterilization containers 30. Other structures may also be positioned between or proximate to either of the first or second valve housing structures 22, 24, such as gaskets, seals, or various other components of the valve 20.

The valve 10 includes a first valve portion 40 and a second valve portion 50, both of which are substantially housed within the valve housing 20. Either one, or both of the first and second valve portions 40, 50 may be movable within the valve housing 20, which generally remains secured to the medical sterilization container 30. For example, most commonly, the first valve portion 40 will be affixed to the valve housing 20 such that it remains substantially stationary, relative to the valve housing 20. In FIG. 2, the first valve portion 40 is illustrated as being connected to the valve housing 20 between the first valve housing structure 22 and the medical sterilization container 30. The first valve portion 40 may extend towards a center of the valve 10, thereby positioning at least a portion of the first valve portion 40 protruding away from the first and second valve housing structures 22, 24. This protruding part of the first valve portion 40 may be used to contact the second valve portion 50 to open or close the valve 10.

The second valve portion 50 may be located proximate to the first valve portion 40, such that the two structures may be moved to contact each other and moved away from contact with each other. Generally, the second valve portion 50 is movable within the valve housing 20, wherein the movement is controlled, at least in part, by the SMA device 60. For example, in FIG. 2, movement of the second valve portion 50 may be along an axis of the valve housing 20, whereby the second valve portion 50 contacts the protruding portion of the first valve portion 40. Other movement of the second valve portion 50 may also occur, such as movement within the shape of the second valve portion 50, lateral movement of the second valve portion 50, or movement in another direction.

The movement of the second valve portion 50 may be between at least two positions: a first position where the second valve portion 50 is in contact with the first valve portion 40, and a second position where the second valve portion 50 is not in contact with the first valve portion 40. In other words, the second valve portion 50 may be moved between a closed valve position (first position) and an open valve position (second position). This movement allows the valve 10 to be open and thus allow material to pass from outside the medical sterilization container 30 to the interior portion 36 (FIG. 1) of the medical sterilization container 30, or be closed and prevent any material or substance from entering or exiting the interior portion 36 medical sterilization container 30.

The movement of the second valve portion 50 is controlled, at least in part, with the SMA device 60, in that, the SMA device 60 moves the second valve portion 50 between the first and second positions. The SMA device 60 is connected to the second valve portion 50, which may include any type of connection, mechanical communication, or other direct or indirect physical contact. The SMA device 60 may also be in contact or mechanical communication with any other structures of the valve 10, including the first or second valve housing structures 22, 24. The SMA device 60 may have a plurality of designs and shapes, such as a biasing structure or spring, as is shown in FIG. 2. For example, in FIG. 2, the SMA device 60 includes a single SMA spring located on one side of the second valve portion 50 and a second SMA spring located on another side of the second valve portion 50. Other shapes of the SMA device 60, such as coned biasing structures or integral sheet structures, as is discussed in additional embodiments herein, may also be included. Furthermore, the SMA device 60 may include any number of separate or unitary parts, such as two biasing structures, or a plurality of SMA devices 60.

The SMA device 60 may be understood as a shape memory alloy structure, which may also include a smart metal, memory metal, memory alloy and smart alloy structure, that can take a position depending on a temperature. The SMA device 60 may function by returning to a remembered, original, or pre-deformed shape from a deformed shape. Often, the original shape is one given to the SMA device 60 when it was cold-forged, and thus corresponds to a cold temperature or low thermal energy of the SMA device 60. In contrast, the deformed shape may correspond to a heated temperature or high thermal energy within the SMA device 60. Therefore, in general terms, the SMA device 60 will be located in one position at a high temperature and another position at a low temperature. The specific temperature for each position may depend on the specific SMA device 60, and may include a variety of different temperatures, all of which are considered within the scope of this disclosure.

It is noted that any of the components of the valve 10 disclosed herein may be constructed from a variety of materials in a variety of methods. For example, the first and second valve portions 40, 50 may be constructed from rubbers, plastics, or other materials capable of forming a seal when in contact. The valve housing 20 may be constructed from a hardened plastic, metal, or other material that substantially resists deformation when subjected to different temperatures or humid environments. The SMA device 60 may be constructed from any material having SMA properties, including but not limited to copper-zinc-aluminum-nickel, copper-aluminum-nickel, and nickel-titanium (NiTi) alloys, as well as various combinations of zinc, copper, gold, and iron alloys.

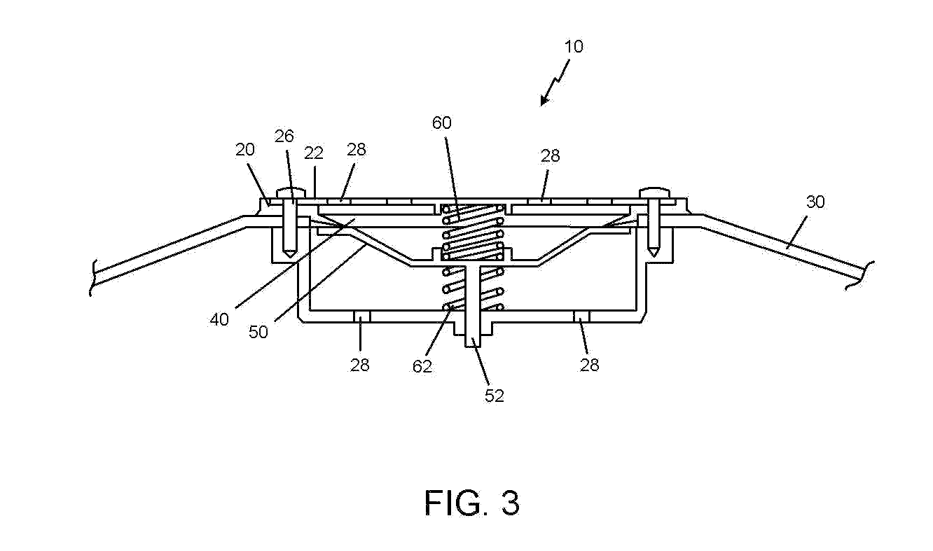

FIG. 3 is a cross-sectional illustration of a medical sterilization container valve 10, in accordance with the first exemplary embodiment of the present disclosure. Specifically, FIG. 3 depicts the second valve portion 50 in contact with the first valve portion 40 to create a sealed or closed valve 10, whereby minimal or no sterilant, fluid or gas may traverse through the valve 10. In other words, when the second valve portion 50 contacts the first valve portion 40, the valve is moved into the open position (first position). Thus, a comparison between FIG. 2 and FIG. 3 illustrates how the second valve portion 50 may move to either be in contact with the first valve portion 40 (FIG. 3), or not be in contact with the first valve portion 40 (FIG. 2).

This movement is controlled by the SMA device 60, and may function as is disclosed in the follow example. A quantity of medical instruments may be positioned within the medical sterilization container 30, which may be closed to substantially seal off the interior portion 36 (FIG. 1). Normally, this is done at room temperature or a low temperature, such that the SMA device 60 is subjected to lower temperatures, and the valve 10 is closed. As is shown in FIG. 3, the SMA device 60, which is located between the second valve portion 50 and the first valve housing structure 22, may be in a contracted position, which places the second valve portion 50 in contact with the first valve portion 40. A spring 62 may also be used to bias the second valve portion 50 towards the first valve portion 40. The spring 62 may be a stainless steel spring, or another biasing device, that biases the valve 10 into the closed position at a low temperature. The SMA device 60 may also include both springs that work together to open and close the valve 10 based on an environmental temperature. The medical sterilization container 30, with the valve 10 in a closed positioned, may then be placed within a sterilization environment, such as an autoclave which has temperatures high enough to sterilize the medical instruments within the medical sterilization container 30.

As the medical sterilization container 30 heats up within the sterilization environment, the SMA device 60 within the valve 10 may also experience a rise in temperature due to the thermal condition within the sterilization environment. As the SMA device 60 approaches a specified temperature, such as 212° F., or any other predetermined temperature, the SMA device 60 may expand and move the second valve portion 50 away from the first valve portion 40. A valve stem 52 may be used to align the second valve portion 50 during movement. The SMA device 60 in this expanded position is shown in FIG. 2. The spring 62 may provide resistance against the SMA device 60, which the SMA device 60 overcomes during its expansion. When the second valve portion 50 is free from contact with the first valve portion 40, an access path may be provided through the valve 10, wherein sterilization material, gas, or other substances may pass through. This allows for the movement of sterilization material into the medical sterilization container 30 to sterilize the medical instruments contained therein. Additionally, this access path allows for the release of pressure from within the medical sterilization container 30.

When the sterilization process finishes, the temperature within the sterilization environment decreases, which lowers the temperature of the valve 10 and changes the thermal condition of the SMA device 60. When the SMA device achieves a certain temperature, it reforms to its original shape, thereby moving the second valve portion 50 into contact with the first valve portion 40. This contact between the first valve portion 40 and the second valve portion 50 creates a biologically impermeable seal between an interior portion 36 (FIG. 1) of the medical sterilization container 30 and an external atmosphere of the medical sterilization container 30. This seal may prevent containments, bacteria, or other harmful substances from contaminating the medical instruments within the medical sterilization container 30. Accordingly, the medical instrument container 30 with the seal formed between the first and second valve portions 40, 50 may be retained without being subject to contamination for any period of time.

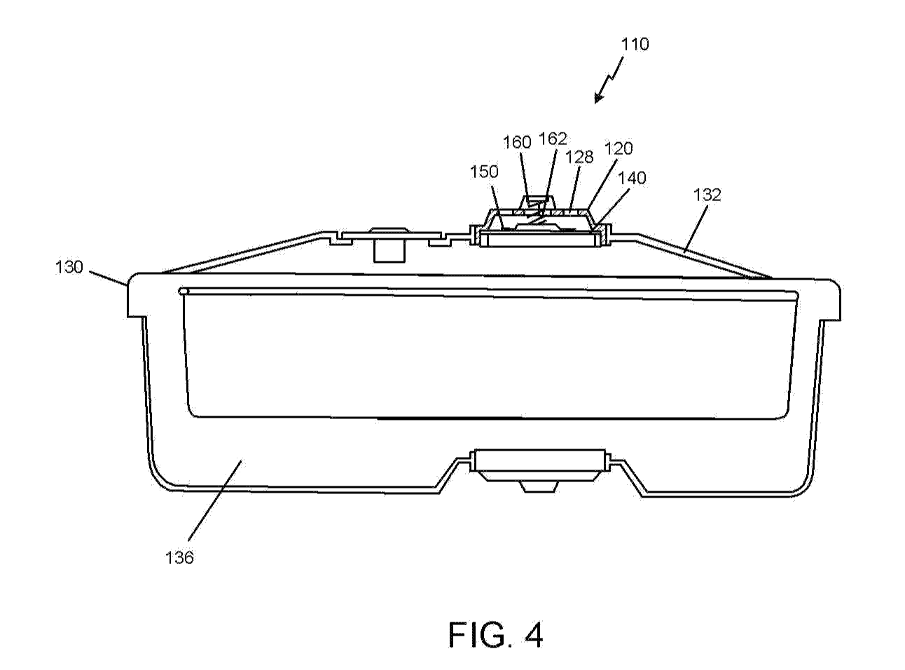

FIG. 4 is a cross-sectional illustration of a medical sterilization container valve 110 within a medical sterilization container 130, in accordance with a second exemplary embodiment of the present disclosure. The medical sterilization container valve 110 (hereinafter, “valve 110”) of the second exemplary embodiment may be substantially similar to the valve 10 of the first exemplary embodiment. Accordingly, any of the structures, features, functions, or characteristics disclosed with respect to the first exemplary embodiment may be included in the second exemplary embodiment.

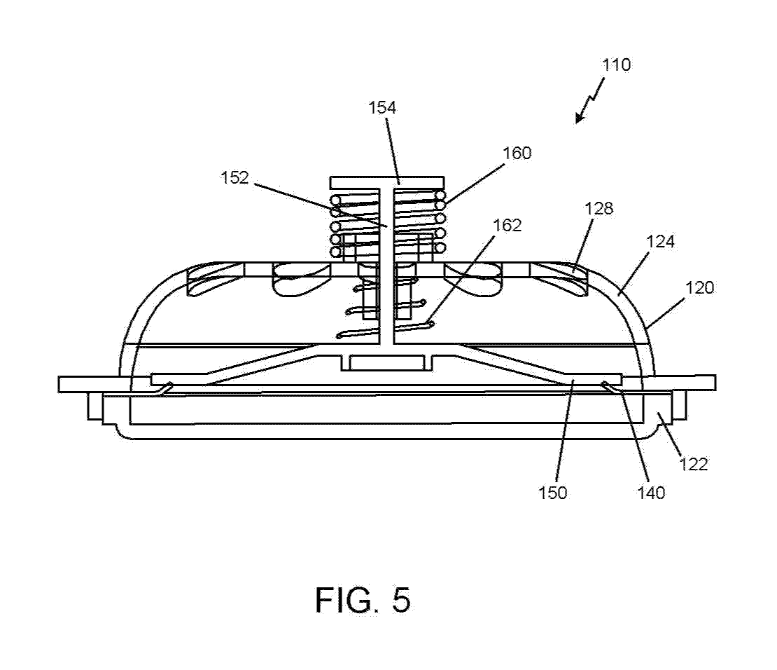

The valve 110 functions similarly to that described of the valve 10 of the first exemplary embodiment. The valve 110 may be located in a portion of the medical sterilization container 130, such as in a cover or a base. More than one valve 110 may be included, depending on the size of the container 130 or the intended use of the medical sterilization container 130. In use, the SMA device 160 may be compressed at a low temperature, such as at room temperature, which may force the valve 110 closed, as illustrated in FIGS. 4 and 5. The closed valve 110 may be further assisted with a vacuum within the medical sterilization container 130. When the medical sterilization container 130 and valve 110 are subjected to high temperatures, such as temperatures above 100° F., above 150° F. or above 212° F., the SMA device 160 may extend, thereby compressing the spring 162 and opening the valve 110 by moving the second valve portion 150 away from the first valve portion 140.

FIG. 5 is a cross-sectional illustration of a medical sterilization container valve 110, in accordance with the second exemplary embodiment of the present disclosure. The valve 110 includes a valve housing 120 that connects to a medical instrument sterilization container 130 (FIG. 4). The valve housing 120 may include perforations 128 for releasing pressure through the medical sterilization container valve 110. The first valve portion 140 is connected to the valve housing 120 and may be contacted by the second valve portion 150 to create a biologically impermeable seal. A SMA device 160 is located proximate to a valve stem 152 and moves the valve stem 152 to move the second valve portion 150. A spring 162 may be included to counteract the force of the SMA device 160.

The valve stem 152 may have an engagement platform 154 connected to the valve stem 152. As is shown, a proximate end of the valve stem 152 may be connected to the second valve portion 150 and a distal end of the valve stem 152 is connected to the engagement platform 154. The valve stem 152 may extend through the second valve housing structure 124, such that the distal end is located external of the valve housing 120. The engagement platform 154 connected to the distal end may provide an abutment for the SMA device 160. For example, when the SMA device 160 is subjected to high temperatures, the SMA device 160 may expand between the engagement platform 154 and the second valve housing structure 124, thereby forcing the engagement platform 154 away from the second valve housing structure 124. This, in turn, moves the second valve portion 150 away from the first valve portion 140 to open the valve 110.

It is noted that the spring 162 may be used to counterbalance the SMA device 160, or bias the second valve portion 150 to contact the first valve portion 140 when the SMA device 160 is not subjected to a high temperature. For example, the spring 162 may be located within the valve housing 120 and surrounding the valve stem 152, whereas the SMA device 160 may be located external of the valve housing 120 and surrounding the valve stem 152. The spring 162 may bias the second valve portion 150 towards the first valve portion 140 during non-heated conditions, thereby retaining the valve 110 in the closed position. When the SMA device 160 is heated, it may provide a force that overcomes the force of the spring 162 to move the second valve portion 150 away from the first valve portion 140.

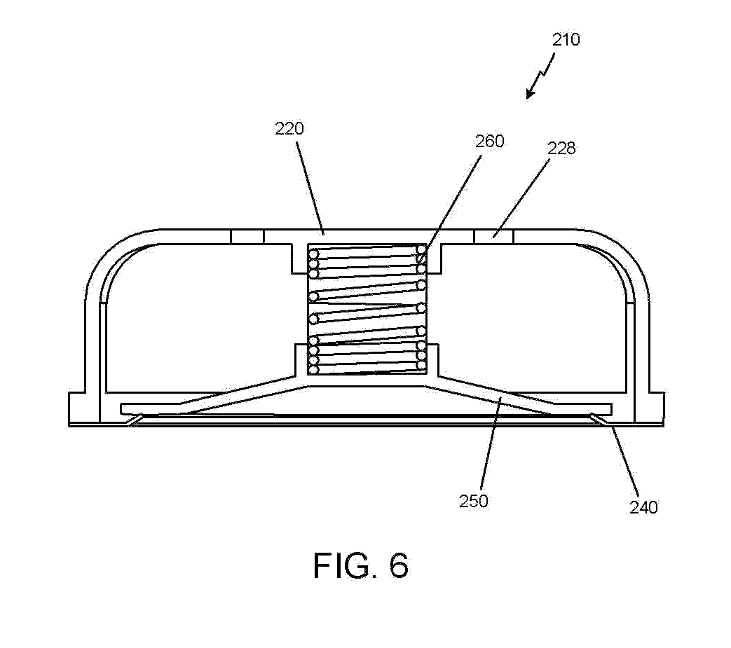

FIG. 6 is a cross-sectional illustration of a medical sterilization container valve 210, in accordance with a third exemplary embodiment of the present disclosure. The valve 210 of the third exemplary embodiment is substantially similar to the valve 10 and valve 110 of the first and second exemplary embodiments, respectively, and may include any of the features, characteristics, functions, or attributes of configurations disclosed in the first and second exemplary embodiments. The valve 210 include an SMA device 260 having a two-way spring, wherein one side of the SMA device 260 is anchored to the valve housing 220 and another side of the SMA device 260 is connected to the second valve portion 250. At a low temperature, the SMA device 260 may extend to close the valve 210 by contacting the second valve portion 250 with the first valve portion 240, as is illustrated in FIG. 6. At a high temperature, the SMA device 260 may compress to open the valve 210, whereby the second valve portion 250 is removed from contacting the first valve portion 240. When the valve 210 is in the open position, with the second valve portion 250 not in contact with the first valve portion 240, equalization of pressure and/or release of a sterilization material may occur through the valve 210, since the valve 210 will allow materials such as steam or sterilization fluid to move through the perforations 228 and the open valve 210.

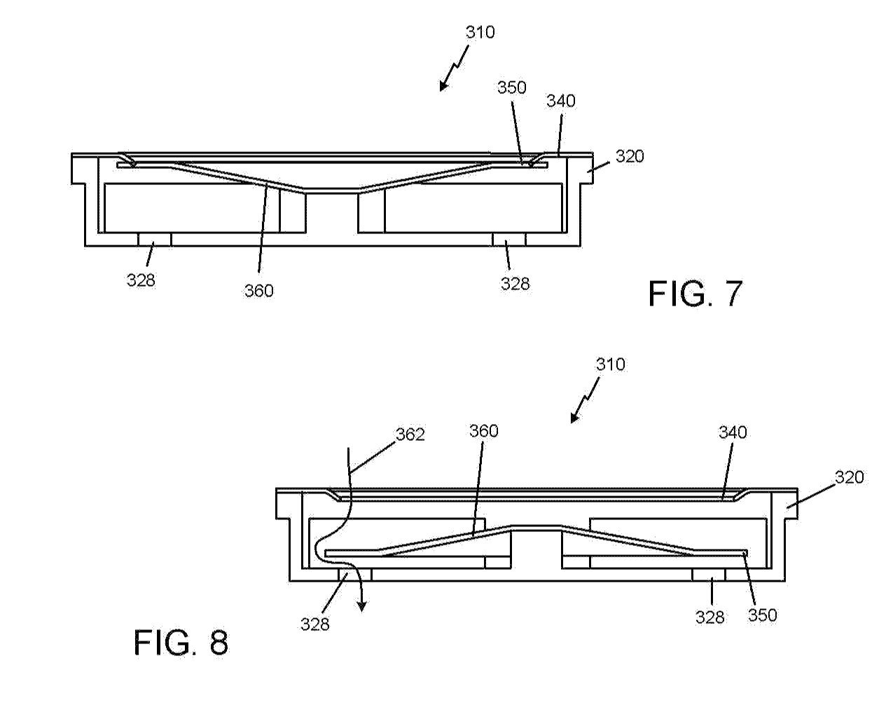

FIG. 7 is a cross-sectional illustration of a medical sterilization container valve 310, in accordance with a fourth exemplary embodiment of the present disclosure. The valve 310 of the third exemplary embodiment is substantially similar to the valve 10, valve 110 and valve 210 of the first, second and third exemplary embodiments, respectively, and may include any of the features, characteristics of configurations disclosed in the first, second and third exemplary embodiments. In FIG. 7, the SMA device 360 is integral with the second valve portion 350. For example, the SMA device 360 may be a two-way trained sheet of SMA material, such as a SMA metal. The SMA device 360 and second valve portion 350 may be affixed to the valve 310, which may be affixed to a medical sterilization container (not shown) by the valve housing 320, which may include a plurality of apertures 328.

FIG. 8 is a cross-sectional illustration of a medical sterilization container valve 310, in accordance with the fourth exemplary embodiment of the present disclosure. The SMA device 360 of FIG. 8 is integral with the second valve portion 350. The SMA device 360 may move from a substantially concave shape, as illustrated in FIG. 7, where the valve 310 is closed, to a substantially convex shape in FIG. 8, where the valve 310 is open. As is indicated in FIG. 8, the opening arrow 362 depicts the open path through the valve 310 and an aperture 328 within the valve 310, when the second valve portion 350 with SMA device 360 is not in contact with the first valve portion 340. This allows the SMA device 360 to open and close the valve 310.

For example, at room temperature or another low temperature, the SMA device 360 may have a concave shape that allows the Second valve portion 350 to contact the first valve portion 340. When the temperature is raised, such as to above 212° F., the SMA device 360 may reverse shape to a convex shape, thereby opening the valve 310, as is illustrated in FIG. 8. This movement of the SMA device 360 may be repeatable for many sterilization cycles thereby allowing the SMA device 360, as well as the SMA devices of other embodiments, to be reused a plurality of times. It is noted that the valve 310 may be designed to be either open or closed with the second valve portion 350 in either a convex or concave shape, or any other shape. For example, the valve 310 may be designed to be in the closed position when the second valve portion 350 is in the convex shape, and in the open position when the second valve portion 350 is in the concave shape.

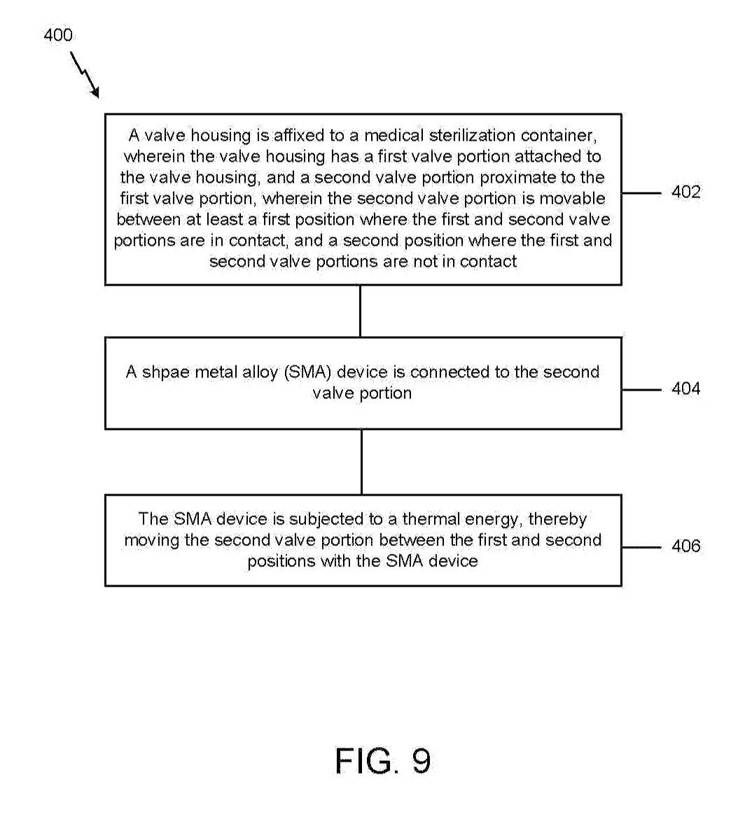

FIG. 9 is a flowchart 400 illustrating a method of utilizing a valve within a medical sterilization container, in accordance with the first exemplary embodiment of the present disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As is shown by block 402, a valve housing is affixed to a medical sterilization container, wherein the valve housing has a first valve portion attached to the valve housing, and a second valve portion proximate to the first valve portion, wherein the second valve portion is movable between at least a first position where the first and second valve portions are in contact, and a second position where the first and second valve portions are not in contact. A shape metal alloy (SMA) device is connected to the second valve portion (block 404). The SMA device is subjected to a thermal energy, thereby moving the second valve portion between the first and second positions with the SMA device (block 406).

The method may also include a number of additional steps or processes, or any variation thereof. Accordingly, the method may include any of the features, functions, or characteristics described with respect to any of the embodiments herein. For example, moving the second valve portion between the first and second positions may include moving the second valve portion into the first position, thereby contacting the first valve portion with the second valve portion, thereby placing the valve in a closed position, and/or moving the second valve portion into the second position, thereby opening the valve by moving the second valve portion out of contact from the first valve portion. The SMA device may be, subjected to a variety of temperatures, and may actuate or engage based on any predetermined temperature. This may include subjecting the SMA device to medical sterilization process having a temperature above 212° F., which may open or close the valve.

In use, the valve within the medical sterilization container may be placed within a sterilizing environment or a sterilization device, wherein the second valve portion moves from the second position to the first positioned while in the sterilization device and while being subjected to a thermal change. The medical sterilization container with the valve house may be removed from within the sterilization device, wherein the second valve portion remains in the first position after removal from within the sterilization device. For example, the SMA device may be subjected to at least a first, a second, and a third temperature, wherein the second temperature is higher than the first and third temperatures. Accordingly, the first temperature may influence the SMA device to place the second valve portion in the first position, the second temperature may influence the SMA device to place the second valve portion in the second position, and the third temperature may influence the SMA device to place the second valve portion in the first position.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

What is claimed is:

1. A medical sterilization container valve comprising:
a valve housing affixed to a medical sterilization container;
a first valve portion attached to the valve housing;
a second valve portion proximate to the first valve portion, wherein the second valve portion is movable between at least a first position and a second position; and
a shape metal alloy (SMA) device connected with the second valve portion, wherein the SMA device moves the second valve portion between the first and second positions.

2. The medical sterilization container valve of claim 1, wherein the valve housing further comprises a first valve housing structure and a second valve housing structure, wherein a portion of the medical sterilization container is positioned between the first valve housing structure and the second valve housing structure.

3. The medical sterilization container valve of claim 2, wherein at least one of the first valve housing structure and the second valve housing structure includes a plurality of perforations.

4. The medical sterilization container valve of claim 1, wherein the SMA device moves the second valve portion between the first and second positions based on a thermal condition of the SMA device.

5. The medical sterilization container valve of claim 1, wherein the first position of the second valve portion places the second valve portion in contact with the first valve portion.

6. The medical sterilization container valve of claim 5, wherein contact between the first valve portion and the second valve portion creates a biologically impermeable seal between an interior portion of the medical sterilization container and an external atmosphere of the medical sterilization container.

7. The medical sterilization container valve of claim 1, wherein the SMA device is connected between the second valve portion and at least one of the first valve housing structure and the second valve housing structure.

8. The medical sterilization container valve of claim 1, wherein the SMA device further comprises at least two SMA biasing structures, wherein one of the at least two SMA biasing structures.

9. The medical sterilization container valve of claim 1, further comprising a valve stem having an engagement platform, wherein a proximate end of the valve stem is connected to the second valve portion and a distal end of the valve stem is connected to the engagement platform, wherein the valve stem is positioned extending through the second valve housing structure with the distal end located external of the valve housing.

10. The medical sterilization container valve of claim 9, further comprising a spring, wherein the spring is located within the valve housing and surrounding the valve stem and the SMA device is located external of the valve housing and surrounding the valve stem, wherein the SMA device contacts the engagement platform.

11. The medical sterilization container valve of claim 1, wherein the SMA device is a two-way SMA biasing device anchored between the second valve portion and the valve housing, wherein the two-way SMA biasing device extends at a first temperature and contracts at a second temperature.

12. The medical sterilization container valve of claim 1, wherein the SMA device further comprises a two-way trained SMA sheet material integrally connected with the second valve portion and substantially forming the second valve portion.

13. The medical sterilization container valve of claim 12, wherein the two-way trained SMA sheet material positions the second valve portion in a concave shape at a first temperature and positions the second valve position in a convex shape at a second temperature.

14. A method of utilizing a valve within a medical sterilization container, the method comprising the steps of:
affixing a valve housing to a medical sterilization container, wherein the valve housing has a first valve portion attached to the valve housing, and a second valve portion proximate to the first valve portion, wherein the second valve portion is movable between at least a first position where the first and second valve portions are in contact, and a second position where the first and second valve portions are not in contact;
connecting a shape metal alloy (SMA) device in to the second valve portion; and
subjecting the SMA device to a thermal energy, thereby moving the second valve portion between the first and second positions with the SMA device.

15. The method of claim 14, wherein moving the second valve portion between the first and second positions further comprises moving the second valve portion into the first position, thereby contacting the first valve portion with the second valve portion, thereby placing the valve in a closed position.

16. The method of claim 14, wherein moving the second valve portion between the first and second positions further comprises moving the second valve portion into the second position, thereby opening the valve by moving the second valve portion out of contact from the first valve portion.

17. The method of claim 14, wherein the step of subjecting the SMA device to the thermal energy further comprises subjecting the SMA device to medical sterilization process having a temperature above 212° F.

18. The method of claim 14, wherein the step of subjecting the SMA device to the thermal energy further comprises:
placing the medical sterilization container with the valve housing within a sterilization device, wherein the second valve portion moves from the second position to the first positioned while in the sterilization device; and
removing the medical sterilization container with the valve house from within the sterilization device, wherein the second valve portion remains in the first position after removal from within the sterilization device.

19. The method of claim 14, wherein the step of subjecting the SMA device to the thermal energy further comprises subjecting the SMA device to at least a first, a second, and a third temperature, wherein the second temperature is higher than the first and third temperatures, wherein the first temperature influences the SMA device to place the second valve portion in the first position, wherein the second temperature influences the SMA device to place the second valve portion in the second position, and wherein the third temperature influences the SMA device to place the second valve portion in the first position.

20. A system for sterilizing medical instruments and storing sterilized medical instruments comprising:
a medical sterilization container having a plurality of side walls and a cover, wherein the cover is openable to expose an interior portion of the medical sterilization container, wherein the interior portion is sized to house a quantity of medical instruments;
a valve positioned within the cover, wherein the valve has a valve housing, a first valve portion attached to the valve housing, and a second valve portion proximate to the first valve portion, wherein the second valve portion is movable between at least a first position and a second position;
a shape metal alloy (SMA) device connected with the second valve portion, wherein the SMA device moves the second valve portion between the first and second positions; and
an access path positioned through the valve between an exterior of the medical sterilization container and the interior portion, wherein the access path is closed when the second valve portion is in the first position and open when the second valve portion is in the second position.
